(19) United States
(12) Patent Application Publication
(10) Pub. No.: US 2022/0160297 A1
(43) Pub. Date: May 26, 2022

(54) SYSTEMS AND METHODS FOR PERSONALIZED LIFESTYLE PROGRAM FOR HEART FAILURE WITH PRESERVED EJECTION FRACTION (HFPEF)

(71) Applicant: Keyto, Inc., San Francisco, CA (US)

(72) Inventors: Ray WU, San Francisco, CA (US); Liane NAKAMURA, Culver City, CA (US)

(21) Appl. No.: 17/535,038

(22) Filed: Nov. 24, 2021

Related U.S. Application Data
(60) Provisional application No. 63/117,711, filed on Nov. 24, 2020.

(51) Int. Cl. A61B 5/00; G16H 50/30; G16H 20/60; G16H 20/30; A61B 5/145; A61B 5/02

(52) U.S. Cl. CPC A61B 5/4833; G16H 50/30; A61B 5/02028; G16H 20/30; A61B 5/14546; G16H 20/60

(57) ABSTRACT

Systems and methods for the implementation of a personalized lifestyle program for the treatment of metabolic-based diseases, such as heart failure with preserved ejection fraction, diabetes-related diseases, neurological disorders, and obesity. As an example, a processor-implemented method is provided for improving disease of a user by personalizing lifestyle program instructions through tolerability and compliance feedback. An example of the processor-implemented method includes: generating, based on a heart failure assessment, a lifestyle program for promoting improvement of heart failure for with preserved ejection fraction including one or more program instructions; generating, based on the one or more program instructions, one or more user interface screens for performing the one or more program instructions through one or more program tasks; and determining a user tolerance of the lifestyle program based on compliance to the performing the one or more program instructions through the one or more program tasks.

### CROSS-REFERENCE TO RELATED APPLICATION(S)

This application claims the benefit of priority to U.S. Provisional Patent Application No. 63/117,711 filed Nov. 24, 2020, the entirety of which is herein incorporated by reference into the Detailed Description below.

### TECHNICAL FIELD

Example embodiments generally relate to systems and methods for implementation of a personalized lifestyle program to improve metabolic-based diseases such as Heart Failure with Preserved Ejection Fraction (HFpEF), diabetes-related diseases and obesity.

### BACKGROUND

Heart failure (HF) is the most common cause of hospitalization in the U.S. for people age >65 years, and as the population ages, the burden of HF will only increase. Half of all patients with HF have a preserved left ventricular (LV) ejection fraction (EF≥50%), which is known as Heart Failure with Preserved Ejection Fraction (HFpEF). HFpEF patients have symptoms and event rates that are equivalent to that of Heart Failure with reduced Ejection Fraction (HFrEF). Mortality rate during hospitalizations for HF is ˜4%, with rates increasing to 10% within one month after discharge. Long term survival is poor, with 5 year survival among patients with HFpEF reported as 35-40% after hospitalization for HF. Because HF also has a substantial impact on quality of life and 20% of men and women past the age of 40 will develop HF, the effect on individuals and cost to society are immense.

HFpEF is certainly a life-threatening and debilitating disease, and there are no proven drugs or devices for HFpEF patients, who are frequently elderly and female, and who commonly have comorbidities such as obesity, hypertension, diabetes, and chronic kidney disease.

The premise behind a ketogenic diet is that by reducing carbohydrate intake to less than 30-50 g/day and replacing those calories with fat, there is a marked reduction in circulating insulin levels limiting fat synthesis/storage and increasing lipolysis (fat breakdown). Evidence suggests that very low-carbohydrate ketogenic diets may have positive effects on weight loss. Ketogenic diets also may have favorable effects on hunger and appetite. Ketogenic diets are unique because the ketone bodies produced can be used as a trackable marker for monitoring and adherence.

Diet and nutrition programs are often difficult for people to consistently follow. People may have difficulty staying motivated over the long period of time needed to achieve a weight loss goal. Daily fluctuations in weight due to factors other than weight loss or gain, such as water loss, may lead to reduced motivation and cessation of dieting. Adherence to a diet requires considerable cognitive and self-regulatory resources at essentially every meal/eating opportunity of every day. Evidence suggests that self-regulatory behavior change strategies, like self-monitoring, play a crucial role in increasing adherence to diet and health behaviors. To address this need a system that gives users real-time feedback on progress and compliance would be effective for adherence and compliance.

Following a ketogenic diet has been shown to be difficult given the restrictiveness it requires. Low-carb diets which allow for more carbohydrate intake than ketogenic can also be effective for weight loss and glycemia control. Furthermore, the elimination of sugar and refined carbs in the presence of medium to high intake of carbohydrates has also been shown to be effective in diet plans such as the Mediterranean diet. Just exercising in the absence of any diet plan has been shown to increase exercise tolerance in patients with HFpEF.

Providing users with a lifestyle and nutrition program that includes the optimally effective instructions that they can tolerate to improve metabolic diseases could benefit many. However, there have been no known examples that do this using a computerized system with assessments and compliance measures by way of program assessment tasks that lead to updated program recommendations.

In addition to HFpEF, there are many other diseases without optimal treatments and that could benefit from a structured lifestyle program. These include HFrEF, non-alcoholic fatty liver disease (NAFLD), non-alcoholic steatohepatitis (NASH), atrial fibrillation, polycystic ovarian syndrome, Alzheimer's disease, Parkinson's, and other neuropsychiatric diseases. They also include ailments such as Type II Diabetes, and obesity.

There is therefore a need to provide a solution for improvement of metabolic-based diseases.

### SUMMARY

According to some example embodiments, systems and methods include implementation of a personalized lifestyle program for the prevention, improvement, or treatment of heart failure with preserved ejection fraction or other metabolic diseases. In some embodiments, there is a first assessment, which may be a heart failure assessment that is used to diagnose or assess severity of heart failure or metabolic disease. In some embodiments, a processor-implemented method generates program instructions based on results from the heart failure assessment for the user. Other assessments that include user demographics and preferences may also be used to generate the program instructions. In some embodiments, it is determined if the user is able to tolerate the provided program instructions through program tasks. The program tasks may be disease specific or related to metabolic health in general. The program instructions may be updated based on the compliance to completing program tasks, results of program tasks, or results of subsequent heart failure assessments.

In some embodiments, the heart failure assessment may include echocardiograms, electrocardiograms, blood tests, exercise capacity assessments such as treadmill tests or cardiopulmonary exercise tests, patient reported outcome measures, or clinical assessments determined by a medical professional such as determination of New York Heart Association (NYHA) class. The heart failure assessment may also be a survey that includes information on demographics, lifestyle and personal preferences.

In some embodiments, ketone measurements are a program task.

In some embodiments, a ketosis score is calculated based on ketone measurements.

In some embodiments, program tasks may include ketone measurements, weight measurements, assessments, surveys, questions, activity monitoring, step count.

In some embodiments, tolerability scores are calculated based on the compliance to completing program tasks, results of the program tasks, or results of heart failure assessments.

In some embodiments, program instructions include level of carbohydrate intake.

In some embodiments, program instructions include activity level, step count, or other exercise recommendations.

In some embodiments, program instructions include food choices, recipes, meal plans, menus, articles, multimedia resources, courses, and lessons.

In some embodiments, program instructions include timing of food intake, which may include time restricted eating windows, intermittent fasting, prolonged fasting, or no restrictions on feeding window.

In some embodiments, low tolerability scores will result in program instructions that are less restrictive than the previous instructions.

In some embodiments, high tolerability scores will result in program instructions that are more restrictive than the previous instructions.

In some embodiments, the program includes rewards for the completion of tasks.

In some embodiments, the program includes lessons, courses, quizzes, questions, ketone measurements, ketosis scores, body weight measurements, exercise goals, activity goals, step count goals, one-to-one messaging, group messaging, community posts, multimedia content.

In some embodiments, heart failure assessments may include lab tests such as insulin level, Homeostatic Model Assessment of Insulin Resistance (HOMA-IR), blood glucose level, Hemoglobin A1C levels.

In some embodiments, heart failure assessments may include patient reported outcome surveys such as the Kansas City Cardiomyopathy Questionnaire (KCCQ), Minnesota Living with Heart Failure (MLHF) survey, or other surveys that assess symptoms, quality of life, and exercise tolerance in users with heart failure.

In some embodiments, weight measurements are a program tasks used to update program instructions.

In some embodiments, ketone measurements are used to update program instructions.

In some embodiments, face-to-face, telephone, or virtual consultation with the user may be used as inputs to tolerability scores.

In some embodiments, the system and methods are configured to promote improvement of heart failure with reduced ejection fraction (HFrEF).

In some embodiments, the system and methods are configured to promote improvement of atrial fibrillation.

In some embodiments, the system and methods are configured to promote improvement of hypertensive heart failure and other types of heart failure.

In some embodiments, the system and methods are configured to promote improvement of non-alcoholic fatty liver disease (NAFLD).

In some embodiments, the system and methods are configured to promote improvement of non-alcoholic steatohepatitis (NASH).

In some embodiments, the system and methods are configured to promote improvement of Alzheimer's disease or other neurologic disorders.

In some embodiments, the system and methods are configured to promote improvement of epileptic diseases.

In some embodiments, the system and methods are configured to promote improvement of coronary artery disease, stroke, or dyslipidemia.

In some embodiments, the system and methods are configured to promote improvement of hypertension.

In some embodiments, the system and methods are configured to promote improvement of kidney disease.

In some embodiments, the system and methods are configured to promote improvement of psychiatric disorders.

In some embodiments, the system and methods are configured to promote improvement of Type I Diabetes.

In some embodiments, the system and methods are configured to promote improvement of Type II Diabetes.

In some embodiments, the system and methods are configured to promote improvement of Metabolic Syndrome.

In some embodiments, the system and methods are configured to promote improvement of Obesity.

### BRIEF DESCRIPTION OF THE DRAWINGS

Example embodiments will now be described, by way of example only, with reference to the accompanying drawings, in which:

FIG. 1 is a flow diagram illustrating a method 100 for generating program instructions, determining the tolerability of the program instructions, and updating the program instructions, according to some embodiments;

FIG. 2 is a diagram illustrating exemplary program instructions that are diet related, according to some embodiments;

FIG. 3 is a diagram illustrating exemplary program instructions that related to what to eat, when to eat, and exercise or activity instructions, according to some embodiments;

FIG. 4 is a flow diagram illustrating a method for determining the tolerability of program instructions, according to some embodiments;

FIG. 5 is a flow diagram illustrating a method for determining the tolerability of program instructions, according to some embodiments;

FIG. 6 shows a system for monitoring and managing a ketosis level of a user, according to some embodiments;

FIG. 7 shows a block diagram of a breath analyzer or breath ketone measurement device for measuring the acetone or ketone in a user's breath, according to some embodiments;

FIG. 8 is an exemplary user interface for providing a user with a ketosis score, according to some embodiments;

FIG. 9 is an exemplary user interface for providing a user with a ketosis score, according to some embodiments;

FIG. 10 is an exemplary user interface for providing a virtual reward, according to some embodiments;

FIG. 11 is an exemplary user interface for providing a user with tasks to earn additional points or virtual currency, according to some embodiments;

FIG. 12 is an exemplary user interface for providing users with additional points or virtual currency for maintain a consecutive streak of performing ketone measurements, according to some embodiments; and

FIG. 13 illustrates a computing device, according to some embodiments.

### DETAILED DESCRIPTION

In the following description of the disclosure and embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

In addition, the term “ketone” is being used broadly to refer to the three endogenously produced ketone bodies: acetone, acetoacetic acid, beta.-hydroxybutyric acid (“β-hydroxybutyrate”), and their conversion equivalents. This term applies regardless of whether the molecule is technically a ketone or whether, for example, it is resonantly equivalent to another class of molecule. For instance, beta.-hydroxybutyric acid (β-hydroxybutyrate), is technically a carboxylic acid rather than a ketone but is nonetheless commonly referred to in the field as a blood ketone.

In addition, the ketone measurement device may also be referred to as a “analyzer,” “device,” or “detector.”

In addition, “points” and “virtual currency” may include numerical values, letters, percentages, real currencies, cryptocurrencies, imaginative currencies, grades, descriptions, stock shares, and or other scales that span from lesser to greater.

Certain aspects of example embodiments include process steps and instructions described herein in the form of an algorithm. It should be noted that the process steps and instructions of example embodiments can be embodied in software, firmware, or hardware and, when embodied in software, can be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

Example embodiments also relate to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, Universal Serial Bus (USB) flash drives, external hard drives, optical disks, compact disc read-only memories (CD-ROMs), magnetic-optical disks, read-only memories (ROMs), random access memories (RAMs), erasable programmable read-only memories (EPROMs), electrically erasable programmable read-only memories (EEPROMs), magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability. The device may include an application (also known as an “App”) which performs the specified commands.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, example embodiments are not described with reference to any particular programming language. It will be appreciated that a variety of programming languages may be used to implement the teachings of example embodiments as described herein.

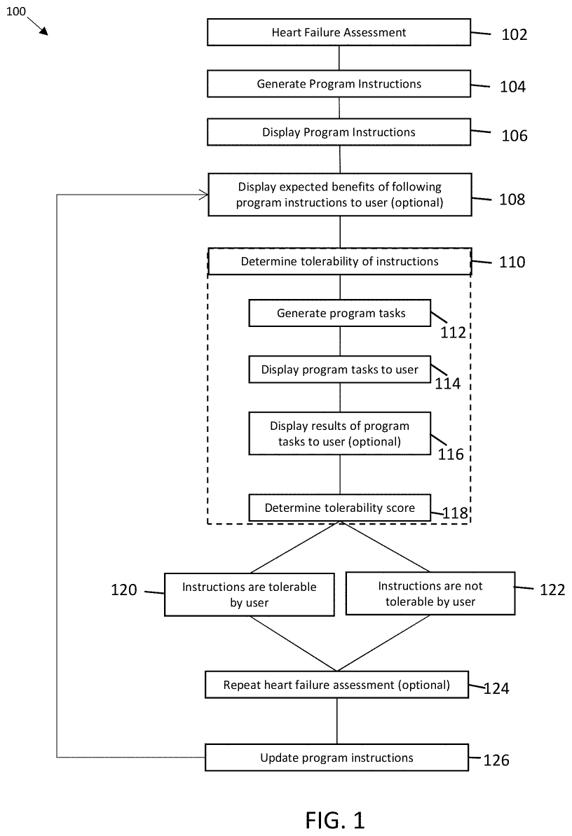

FIG. 1 is a flow diagram illustrating a method 100 for generating a personalized program for the user, who has a disease such as HFpEF. The program may also be personalized for the treatment of Type II diabetes, pre-diabetes, obesity, or other metabolic diseases.

At step 102, a first assessment, which is a heart failure assessment, is initialized. In some embodiments, this assessment may include a diagnostic test such as a blood test, an echocardiogram, an exercise tolerance test, a cardiopulmonary exercise test, magnetic resonance image (MRI), or a ketone measurement. In some embodiments, this assessment may include a patient reported outcome survey, which may include Kansas City Cardiomyopathy Questionnaire, Minnesota Living with Heart Failure (MLHF) survey, or other surveys that assess symptoms, quality of life, and exercise tolerance in users with heart failure or other metabolic-based diseases. The assessment may also include survey questions that may include questions on demographics, lifestyle and personal preferences.

Another example embodiment is system for promoting improvement of non-alcoholic fatty liver disease (NAFLD), the system comprising: one or more processors configured to: generate, based on a NAFLD assessment, a lifestyle program for promoting improvement of NAFLD including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of non-alcoholic steatohepatitis (NASH), the system comprising: one or more processors configured to: generate, based on a NASH assessment, a lifestyle program for promoting improvement of NASH including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of polycystic ovarian syndrome (PCOS), the system comprising: one or more processors configured to: generate, based on a PCOS assessment, a lifestyle program for promoting improvement of PCOS including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of Alzheimer's disease (AD) and other neurologic disorders, the system comprising: one or more processors configured to: generate, based on a neurologic or cognitive assessment, a lifestyle program for promoting improvement of cognition and neurologic function including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of atrial fibrillation (AF), the system comprising: one or more processors configured to: generate, based on a AF assessment, a lifestyle program for promoting improvement of AF including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of heart failure with reduced ejection fraction (HFrEF), the system comprising: one or more processors configured to: generate, based on a HFrEF assessment, a lifestyle program for promoting improvement of HFrEF including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of hypertensive heart failure and other types of heart failure and cardiomyopathies, the system comprising: one or more processors configured to: generate, based on a heart failure assessment, a lifestyle program for promoting improvement of heart failure including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of coronary artery disease, stroke, or dyslipidemia, the system comprising: one or more processors configured to: generate, based on a cardiovascular assessment, a lifestyle program for promoting improvement of cardiovascular function and lipid levels including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of kidney disease, the system comprising: one or more processors configured to: generate, based on a kidney assessment, a lifestyle program for promoting improvement of kidney function and lipid levels including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of hypertension, the system comprising: one or more processors configured to: generate, based on a blood pressure assessment, a lifestyle program for promoting improvement of blood pressure including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of epilepsy causing disorders, the system comprising: one or more processors configured to: generate, based on a epilepsy assessment, a lifestyle program for promoting improvement of symptoms including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of psychiatric disorders such as depression, anxiety, schizophrenia, the system comprising: one or more processors configured to: generate, based on a psychiatric assessment, a lifestyle program for promoting improvement of mood and symptoms including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks.

Another example embodiment is system for promoting improvement of Type II diabetes or pre-diabetes, the system comprising: one or more processors configured to: generate, based on a diabetes assessment, a lifestyle program for promoting improvement of symptoms including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks. For example, in a randomized controlled trial (1,2), incorporated by reference herein in their entirety, the Keyto (R) program of example embodiments and originating from the inventors included a personalized program for pre-diabetes and obesity.

Another example embodiment is system for promoting improvement of obesity, the system comprising: one or more processors configured to: generate, based on an obesity assessment, a lifestyle program for promoting improvement of symptoms including one or more program instructions; generate, based on the one or more program instructions, one or more user interface screens for performing the one or more program instructions through one or more program tasks; and determine a user tolerance of the lifestyle program based on compliance to the performing the one or more program instructions through the one or more program tasks. For example, in a randomized controlled trial (1,2), the Keyto program of example embodiments and originating from the inventors included a personalized program for pre-diabetes and obesity.

One example of the first assessment was reported in a randomized trial comparing the Keyto program vs the Weight Watchers (R) (WW (R)) Program (1,2), herein incorporated by reference in their entity. The Keyto program of example embodiments is studied in those trials. In the Keyto program, users received blood tests and initial weights before getting a personalized program through the App for the treatment of obesity and metabolic diseases.

In some embodiments, the heart failure assessment may be a survey or patient reported outcome survey that is performed on an electronic device. In some other embodiments, the survey is performed on paper and the results may be inputted to an electronic device.

In some embodiments, the heart failure assessment includes diagnostic tests such as laboratory blood tests, echocardiograms, exercise tolerance test, cardiopulmonary exercise test or an MRI. In some embodiments, a clinical diagnoses performed by a medical professional may be part of the heart failure assessment. The results of any of these tests may be inputted into an electronic device.

In some embodiments, the heart failure assessment may include a ketone measurement. This may be performed by a breath sensor, blood sensor, urine sensor, and the results may be wirelessly transmitted to an electronic device or the results may be manually inputted.

One example is from the Keyto vs Weight Watchers trial (1,2). The Keyto program included ketone measurements through a breath sensor.

At step 104, in some embodiments, program instructions are generated based on the assessment results by an electronic device containing a processor.

In some embodiments, the program instructions are generated by previously determined order, decision tree protocol, or machine learning techniques include neural networks, random forest, collaborative filtering, support vector networks.

In some embodiments, the program instructions include recommendations on the type of foods for the user to eat.

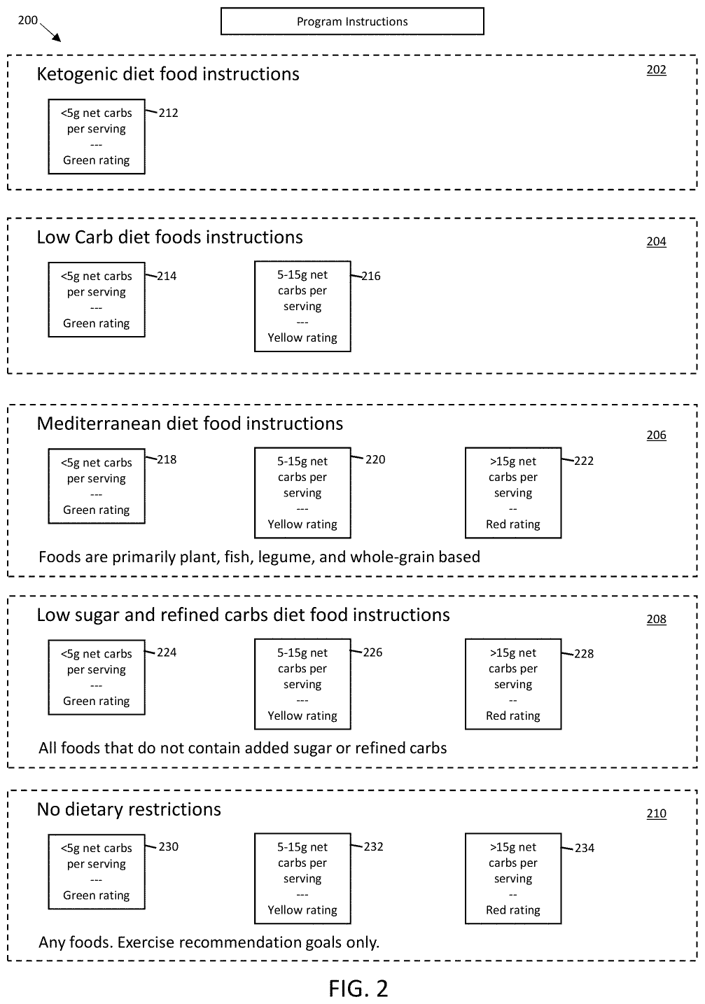

FIG. 2 is a diagram that shows an exemplary set of instructions 200 that may be generated for the user.

The instructions may include that the user consume foods as part of a ketogenic diet 202, which consists of foods that are low in carbohydrates 212. In some embodiments, low carbohydrate foods have less than 5 grams of carbohydrates, or net carbohydrates, per serving. In some embodiments, foods that are low in carbohydrates are given a green rating on the App

The instructions may include that the user consume foods as part of a Low-Carb diet 204, which consists of foods that are low in carbohydrates 214 and foods that have a medium amount of carbohydrates 216. Foods that are low in carbohydrates 214 may have less than 5 grams of carbohydrates, or net carbohydrates, per serving, and may have a green rating on the App. Foods that are medium in carbohydrates 216 may have 5-15 grams of carbohydrates, or net carbohydrates, per serving, and may have a yellow rating on the App.

The instructions may include that the user consume foods as part of a Mediterranean diet 206, which consists of foods that are low in carbohydrates 218 and foods that have a medium amount of carbohydrates 220 and foods that have a high amount of carbohydrates 222. Foods that are low in carbohydrates 218 may have less than 5 grams of carbohydrates, or net carbohydrates, per serving, and may have a green rating on the App. Foods that are medium in carbohydrates 220 may have 5-15 grams of carbohydrates, or net carbohydrates, per serving, and may have a yellow rating on the App. Foods that are high in carbohydrates 222 may have greater than 15 grams of carbohydrates, or net carbohydrates, per serving, and may have a red rating on the App. The food recommendations for the Mediterranean diet may include primarily plant based foods, fish based foods, legumes, whole grains, and other foods that are emphasized in a Mediterranean-based diet.

The instructions may include that the user consume foods as part of a low sugar and low refined carb based diet 208, which consists of foods that are low in carbohydrates 224 and foods that have a medium amount of carbohydrates 226 and foods that have a high amount of carbohydrates 228. Foods that are low in carbohydrates 224 may have less than 5 grams of carbohydrates, or net carbohydrates, per serving, and may have a green rating on the App. Foods that are medium in carbohydrates 226 may have 5-15 grams of carbohydrates, or net carbohydrates, per serving, and may have a yellow rating on the App. Foods that are high in carbohydrates 228 may have greater than 15 grams of carbohydrates, or net carbohydrates, per serving, and may have a red rating on the App. The food instructions for the low sugar low refined carb diet may include all foods that do not contain a high amount of sugar, added-sugar, refined carbohydrates.

The instructions may include that the user consume foods without dietary restrictions 210, which consists of foods that are low in carbohydrates 230 and foods that have a medium amount of carbohydrates 232 and foods that have a high amount of carbohydrates 234. Foods that are low in carbohydrates 230 may have less than 5 grams of carbohydrates, or net carbohydrates, per serving, and may have a green rating on the App. Foods that are medium in carbohydrates 232 may have 5-15 grams of carbohydrates, or net carbohydrates, per serving, and may have a yellow rating on the App. Foods that are high in carbohydrates 234 may have greater than 15 grams of carbohydrates, or net carbohydrates, per serving, and may have a red rating on the App. There may not be any restrictions to foods that the user can consume in this diet. Rather, the user is given instructions for exercise or activity only.

An exemplary example of the instructions given to users were described in the Keyto vs WW trial (1,2), as part of the Keyto program.

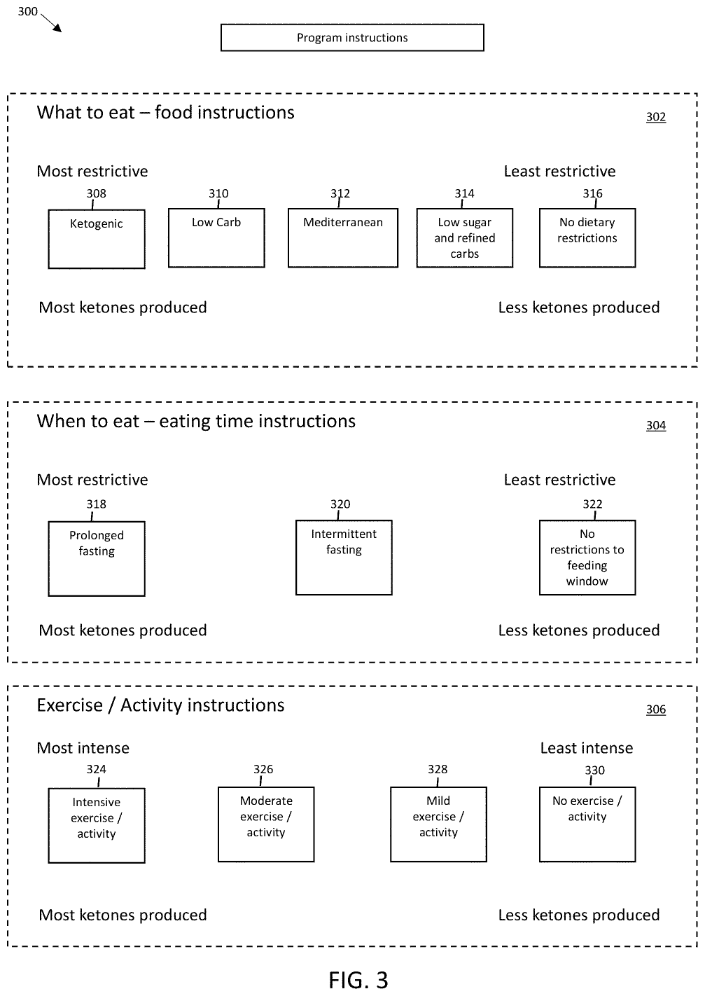

FIG. 3 is a diagram that shows an exemplary set of instructions 300 that may be generated for the user including food instructions 302, as well as instructions for eating time or when to eat (eating time instructions 304) and exercise or activity instructions 306. Instructions may also be given for the amount of food to eat (not shown).

In some embodiments, food instructions 302 may fall on a spectrum of most restrictive such as a ketogenic diet 308, to least restrictive diet such as a diet with no restrictions 316. There may be diets in between such as a low carb diet 310, Mediterranean diet 312, and low sugar and refined carb diet 314.

The most restrictive diets may enable the user to produce the most ketones, which may have the most positive benefit on heart failure symptoms and other metabolic based disease symptoms. Eating fewer carbohydrates may result in weight loss, improvement in blood sugar levels, improvement of hemoglobin A1C levels, improvement in insulin resistance, and the production of ketones by the liver. The production of ketones may result in increased levels of satiety, and improvement in heart failure as ketones may be an alternative fuel source for the heart. Ketones may also improve cognitive function and subjective mood. The production of ketones by the liver may reduce fat and fibrosis in the liver and the heart.

In some embodiments, the program instructions may include the use of exogenous ketones in the form of ketone salts or ketone esters, medium chain triglycerides, short chain triglycerides (not shown). The use of these supplements or pharmaceuticals may aid in increased production of ketones or increased levels of ketones in the user's body, which may improve heart, brain, or other body organ functions.

In some embodiments, eating time instructions 304 may fall on a spectrum of most restrictive such as a prolonged fasting 318, to least restrictive such as eating with no restrictions on feeding window 322. There may be eating in between such as intermittent fasting 320. The most restrictive eating windows may enable the user to produce the most ketones.

In some embodiments, exercise or activity instructions 306 may fall on a spectrum of most intense such as intensive exercise 324, to least intense such no exercise or activity recommendations 330. There may be exercise or activity instructions in between such as moderate activity 326 or mild activity 328. The most intense exercise or activity may enable the user to produce the most ketones and improve cardiovascular health and exercise capacity to the greatest degree.

In some embodiments, instructions on the amount of food to eat (not shown) may include the amount of carbohydrates to eat per day, the amount of calories to eat per day, or the amount of satiety to eat per meal. In some embodiments, the number of green rated foods, yellow rated foods, or red rated foods may be given.

Returning to FIG. 1, after the program instructions are generated at step 104, the instructions are displayed to the user at step 106. In some embodiments, the instructions may be displayed on a mobile application (App) on a portable electronic device. In some other embodiments, the instructions may displayed be on a display, e.g., monitor from a computer or the screen of a laptop.

In some embodiments, the expected benefits of following the instructions may be displayed at step 108. These expected benefits may include in some embodiments an improvement in symptoms from heart failure such as shortness of breath, swelling, edema, night time comfort, dyspnea, exercise tolerance or other symptoms. In some embodiments, they may include weight loss or improvement in energy, sleep, mental clarity, or other wellness related attributes. In some embodiments, they may include the accomplishment of goals set by the user themselves such as walking a race, or attending a grandchild's wedding.

In some embodiments, the method 100 then determines at step 110 the tolerability of the instructions generated.

In some embodiments, this is accomplished by generating program tasks at step 112, displaying the program tasks to the user at step 114 and determining a tolerability score based on the responses or compliance to the program tasks at step 118.

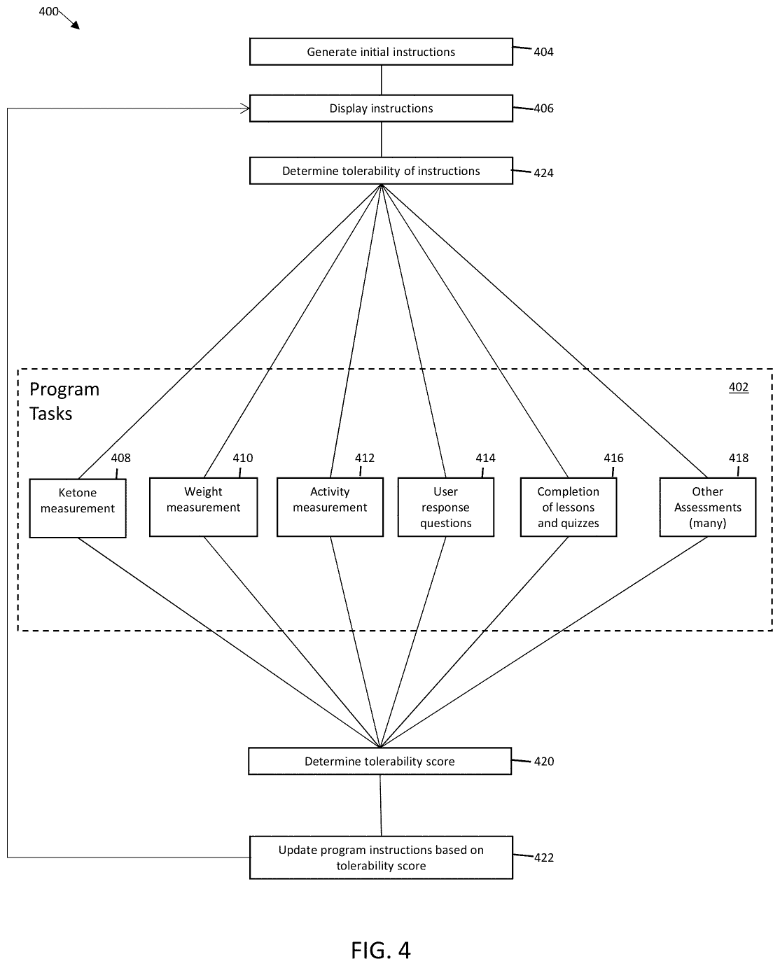

FIG. 4 is a flow diagram of a method 400 that shows an exemplary set of program tasks 402 that may be assigned to the user.

In some embodiments, the initial program instructions are generated for the user step 404, and the program instructions are displayed the user step 406. The program tasks 402 may be used to determine the tolerability of the program instructions 424.

In some embodiments, program tasks may include compliance tasks such as ketone measurements 408, weight measurements 410, activity measurements 412, responses to questions on the app or on other electronic devices 414, or completion of courses, lessons and quizzes 416.

In some embodiments, other assessments 418 may be included as program tasks.

These may include heart failure assessments, which may be diagnostic tests such as a blood test, an echocardiogram, an exercise tolerance test, a cardiopulmonary exercise test, an MRI, or a ketone measurement. In some embodiments, this assessment may include a patient reported outcome survey, which may include Kansas City Cardiomyopathy Questionnaire, Minnesota Living with Heart Failure (MLHF) survey, or other surveys that assess symptoms, quality of life, and exercise tolerance in users with heart failure or other metabolic-based diseases.

In some embodiments, the App can provide users with the ability to receive points or virtual currency for completing program tasks. Users may receive more points or virtual currency for better performance of the tasks, such as earning more points for higher ketosis levels than lower ketosis levels, or more weight loss than less weight loss.

In some embodiments, the points or virtual currency may be redeemed for virtual rewards such as badges, rankings, and software features, or tangible rewards such as money, gift cards, or other prizes.

In any of the embodiments, the program task results may be displayed on the display to the user on the App.

In some embodiments, the compliance tasks may be used to determine a tolerability score 420. For example, for ketone measurements, 408 which may be performed from a breath measurement, blood measurement, or urine measurement, the frequency of measurement and ketosis level detected may be used as inputs to determining the tolerability score. For weight measurements 410, the frequency of measurement and weight change may be used as inputs to determining the tolerability score. For activity measurement 412, the amount of activity performed may be used as inputs to determining the tolerability score. For user response questions 414, the answers to the questions may be used as inputs to determining the tolerability score. For lessons and quizzes, the completion of lessons and quiz results may be used as inputs to determining the tolerability score. For other assessments, the respective results may be used as inputs to determining the tolerability score.

In some embodiments, the tolerability score may be determined by algebraic functions, stochastic functions, decision tree protocols, or machine learning techniques which may include neural networks (including deep neural networks, DNN), random forest, collaborative filtering, support vector networks.

In some embodiments, the heart failure or other disease assessments may be performed after the tolerability scores are determined. This is exemplified in FIG. 1 step 124.

A neural network consists of neurons. A neuron is a computational unit that uses xₛ and an intercept of 1 as inputs. An output from the computational unit may be:

h_{W,b}(x)=ƒ(W^{T}x)=ƒ(Σₛ₌₁ⁿWₛxₛ+b)

s=1, 2, . . . n, n is a natural number greater than 1, Wₛ is a weight of xₛ, b is an offset (i.e. bias) of the neuron and f is an activation function (activation functions) of the neuron and used to introduce a nonlinear feature to the neural network, to convert an input of the neuron to an output. The output of the activation function may be used as an input to a neuron of a following convolutional layer in the neural network. The activation function may be a sigmoid function. The neural network is formed by joining a plurality of the foregoing single neurons. In other words, an output from one neuron may be an input to another neuron. An input of each neuron may be associated with a local receiving area of a previous layer, to extract a feature of the local receiving area. The local receiving area may be an area consisting of several neurons.

A deep neural network (DNN) is also referred to as a multi-layer neural network and may be understood as a neural network that includes a first layer (generally referred to as an input layer), a plurality of hidden layers, and a final layer (generally referred to as an output layer). A layer is considered to be a fully connected layer when there is a full connection between two adjacent layers of the neural network. To be specific, all neurons at an i^{th} layer is connected to any neuron at an (i+1)^{th} layer. In the DNN, more hidden layers enable the DNN to depict a complex situation in the real world. Training of the deep neural network is a weight matrix learning process. A final purpose of the training is to obtain a trained weight matrix (a weight matrix consisting of learned weights W of a plurality of layers) of all layers of the deep neural network.

The neural network can be trained using labelled data pairings between one or more inputs and one or more outputs. For example, the labelled data pairings can include inputs of the program task assessments and outputs of, either i) tolerability score, ii) program instructions, or iii) individual respective tolerability score of each program task assessment.

In some embodiments, the tolerability score may be determined over the course of days. In other embodiments, it may be determined over the course of weeks. In other embodiments, it may be determined over the course of months. In other embodiments, it may be determined over the course of years.

In some embodiments, the tolerability score is used to update the program recommendations 422.

In some embodiments, the program instructions may be updated based on algebraic functions, stochastic functions, decision tree protocols, or machine learning techniques which may include neural networks, random forest, collaborative filtering, support vector networks.

As above, the program instructions may be generated from a neural network based model. The neural network can be trained using labelled data pairings between one or more inputs and one or more outputs. For example, the labelled data pairings can include inputs of the heart failure assessments, program tasks and outputs of program instructions, which may include what to eat, when to eat, and exercise/activity instructions.

In some embodiments, the updated program recommendations are displayed on the display to the user 406.

In some embodiments, the tolerability of the updated instructions 424 as determined through the program tasks 402 is repeated.

In some embodiments, this continues to repeat indefinitely while the user remains on the program. In some embodiments, this duration could weeks, months, or years.

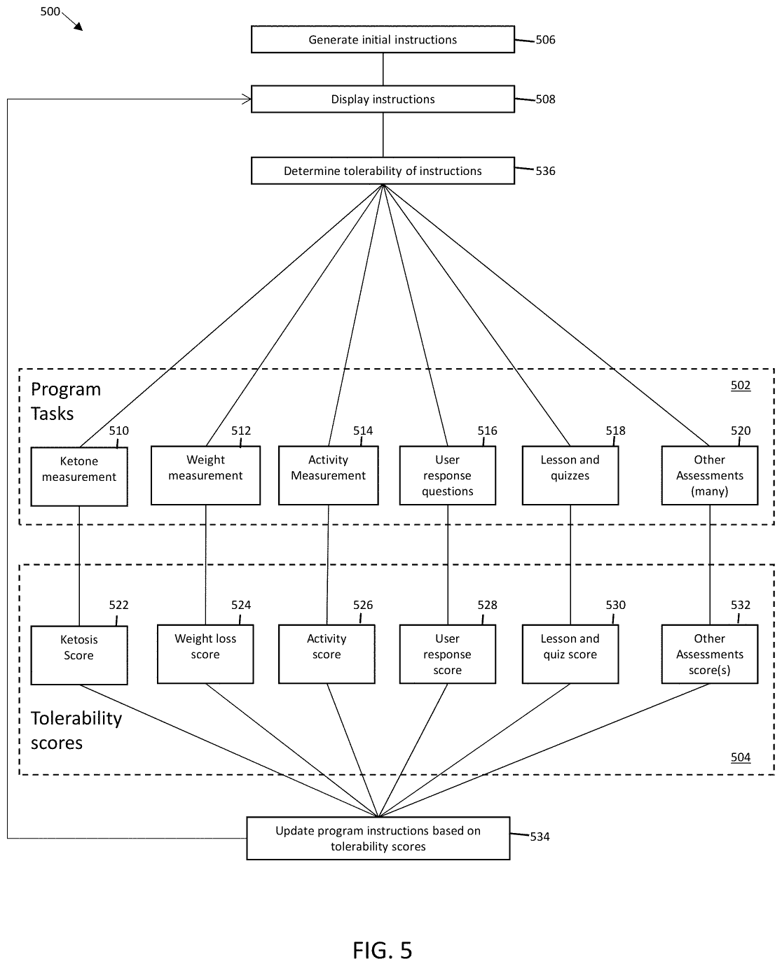

FIG. 5 is another flow diagram of a method 500 that shows another embodiment for how program instructions may be updated by determining tolerability of instructions 536 based on program tasks 502.

In some embodiments, the initial program instructions are generated for the user step 506, and the program instructions are displayed the user step 508. The program tasks 502 may be used to determine the tolerability of the program instructions 536.

In some embodiments, program tasks may include compliance tasks such as ketone measurements 510, weight measurements 512, activity measurements 514, responses to questions on the App or on other electronic devices 516, or completion of courses, lessons and quizzes 518.

In some embodiments, other assessments 520 may be included as program tasks. These may include heart failure assessments, such as diagnostic tests such as a blood test, an echocardiogram, an exercise tolerance test, a cardiopulmonary exercise test, an MRI, or a ketone measurement. In some embodiments, this assessment may include a patient reported outcome survey, which may include Kansas City Cardiomyopathy Questionnaire, Minnesota Living with Heart Failure (MLHF) survey, or other surveys that assess symptoms, quality of life, and exercise tolerance in users with heart failure or other metabolic-based diseases.

According to some embodiments, the program tasks 502 may be used to determine tolerability scores 504 which may include ketosis score of step 522, which may be based on ketone measurements 510. Weight loss score 524 which may be based on weight measurements 512. Activity score 526 which may be based on activity measurements 514. User response score 528 which may be based on User response questions 516. Lesson and quiz score 530 which may be based on lesson and quizzes 518. Other assessment scores 532 which may include many scores based on many other assessments 520.

In some embodiments, the heart failure or other disease assessments may be performed after the tolerability scores are determined. This is exemplified in FIG. 1 step 124.

In some embodiments, the user is given a goal for each of the program tasks 502. In some embodiments, the goals are displayed on the App. One example of a goal can be a certain amount of weight to lose. Another example is to maintain a certain ketosis score. Another example is to perform ketone measurements with a certain frequency. Another example is to perform a certain number of steps per day. Another is to improve a heart failure assessment metric score by a specified range.

In some embodiments, the App can provide users with the ability to receive points or virtual currency for completing program tasks. Users may receive more points or virtual currency for better performance of the tasks, such as earning more points for higher ketosis levels than lower ketosis levels, or more weight loss than less weight loss.

In some embodiments, the points or virtual currency may be redeemed for virtual rewards such as badges, rankings, and software features, or tangible rewards such as money, gift cards, or other prizes.

According to some embodiments, at step 522, the App determines a ketosis score based on the acetone measurement data received from the ketone analyzer, beta-hydroxybutrate measurement data received from a blood ketone monitor, or acetoacetate measurement received from a urine ketone monitor.

According to some embodiments, the ketosis score is generated by converting the raw data received from a breath analyzer to a value associated with predefined measurement scale. For example, the raw data may be converted to parts-per-million (PPM) values. The score may then be determined by comparing the PPM value(s) or to values generated from the PPM values to predetermined thresholds. In some embodiments, the score is a number associated with a predefined level of acetone in the user's breath. The score may be a number from 1-3, 1-5, 1-6, 1-8, 1-10, 1-15, 1-20, 1-50, or any other suitable range of numbers. Each score may be associated with lower and upper thresholds. For example, a score of 1 may be associated with PPM values that are below 5 PPM, a score of 2 may be associated with PPM values from 5 to 10, a score of 3, may be associated with PPM values from 10-15, and so on. In some embodiments, the scores are qualitative, rather than quantitative. For example, a range of scores may be low, moderate, high, and very high.

According to some embodiments, the ketosis score is generated by using blood ketone values. For example, the score may be determined by comparing the mmol/L value(s) to predetermined thresholds. The score may be a number from 1-3, 1-5, 1-6, 1-8, 1-10, 1-15, 1-20, 1-50, or any other suitable range of numbers. Each score may be associated with lower and upper thresholds. For example, a score of 1 may be associated with mmol/L values that are below 0.5 mmol/L, a score of 2 may be associated with mmol/L values from 0.5 to 1.0, a score of 3, may be associated with mmol/L values from 1.0-1.5, and so on. In some embodiments, the scores are qualitative, rather than quantitative. For example, a range of scores may be low, moderate, high, and very high. In some embodiments, the scores are in the original units of the measurement, such as mmol/L.

According to some embodiments, the ketosis score is generated by using urine ketone values. For example, the score may be determined by comparing the mg/dl value(s) to predetermined thresholds. The score may be a number from 1-3, 1-5, 1-6, 1-8, 1-10, 1-15, 1-20, 1-50, or any other suitable range of numbers. Each score may be associated with lower and upper thresholds. For example, a score of 1 may be associated with mg/dl values that are below 5 mg/dl, a score of 2 may be associated with mg/dl values from 5 to 15, a score of 3, may be associated with mg/dl values from 15-40, and so on. In some embodiments, the scores are qualitative, rather than quantitative. For example, a range of scores may be low, moderate, high, and very high. In some embodiments, the scores are in the original units of the measurement, such as mg/dl.

The thresholds defining scores can be based on clinical data associated with ketone levels for test subjects undergoing various levels of ketosis. For example, a ketosis score of “low” or a low number may be associated with ketone levels for test subjects who recently consumed carbohydrates and, thus, are likely metabolizing little if any fat. A score of “very high” or a high number may be associated with ketone levels for test subjects that have high levels of ketones in their bloodstream.

According to some embodiments, the App determines a score for a given breath analysis by comparing the acetone measurement or a value that is based on the acetone measurement, such as the PPM value or a value based on the PPM value, to the thresholds defining the scores. For example, a PPM value of 10 that results from a breath acetone measurement may correspond to a “moderate” score.

In some embodiments, individual ketosis scores may be combined and analyzed with statistical, stochastic, algebraic, and machine learning techniques to determine an overall ketosis score. As above, the individual ketosis scores may be generated from a neural network based model. For example, the labelled data pairings can include inputs of the ketosis measurement levels and frequencies (from one or multiple users) and outputs of a ketosis score, which may be a numerical value, or a qualitative value.

In some embodiments, weight measurements 512 may be used to determine a weight loss score 524. This weight loss score may be based on multiple weight measurements and determined by statistical, stochastic, algebraic, and machine learning techniques to determine an overall ketosis score. As above, the weight measurements 512 may be generated from a neural network based model. For example, the labelled data pairings can include inputs of the weight measurement levels and frequencies (from one or multiple users) and outputs of a weight loss score, which may be a numerical value, or a qualitative value.

In some embodiments, activity measurements 514 may be used to determine a activity score 526. This activity score may be based on multiple activity measurements and determined by statistical, stochastic, algebraic, and machine learning techniques to determine an overall ketosis score. As above, the activity measurements 514 may be generated from a neural network based model. For example, the labelled data pairings can include inputs of the activity measurement levels and frequencies (from one or multiple users) and outputs of a activity score, which may be a numerical value, or a qualitative value.

In some embodiments, user response questions 516 may be used to determine a user response score 528. This user response score may be based on multiple user response questions and determined by statistical, stochastic, algebraic, and machine learning techniques to determine an overall ketosis score. As above, the user response questions 516 may be generated from a neural network based model. For example, the labelled data pairings can include inputs of user responses (from one or multiple users) and outputs of a user response score, which may be a numerical value, or a qualitative value.

In some embodiments, lesson and quizzes 518 may be used to determine a lesson and quiz score 530. This weight loss score may be based on multiple lesson and quiz results and determined by statistical, stochastic, algebraic, and machine learning techniques to determine an overall ketosis score. As above, the lesson and quizzes 518 may be generated from a neural network based model. For example, the labelled data pairings can include inputs of lesson and quiz scores (from one or multiple users) and outputs of a lesson and quiz score, which may be a numerical value, or a qualitative value.

In some embodiments, other assessments 520 may be used to determine other assessment scores 532. There may be numerous other assessment scores. These other assessment scores may be based on multiple other assessment results and determined by statistical, stochastic, algebraic, and machine learning techniques to determine an overall ketosis score. As above, the other assessments may be generated from a neural network based model. For example, the labelled data pairings can include inputs other assessment results (from one or multiple users) and outputs of a other assessment score, which may be a numerical value, or a qualitative value.

In any of the embodiments, the program task scores may be displayed to the user on the App.

In some embodiments, the tolerability scores 504 may be determined over the course of days. In other embodiments, it may be determined over the course of weeks. In other embodiments, it may be determined over the course of months. In other embodiments, it may be determined over the course of years.

In some embodiments, the tolerability scores 504 are combined into an overall tolerability score (not shown).

In some embodiments, the tolerability scores 504 are used to update the program recommendations 534.

In some embodiments, the program instructions may be updated based on algebraic functions, stochastic functions, decision tree protocols, or machine learning techniques which may include neural networks, random forest, collaborative filtering, support vector networks. For example, the labelled data pairings can include inputs of the ketosis score, weight loss score, activity score, user response score, lesson and quiz score, other assessment score (all from one or multiple users) and outputs of outputs of program instructions, which may include what to eat, when to eat, and exercise/activity instructions.

In some embodiments, the updated program instructions are displayed to the user 508.

In some embodiments, the tolerability of the updated instructions 536 as determined by program tasks 502 is repeated.

In some embodiments, this process continues to repeat indefinitely while the user remains on the program. In some embodiments, this duration could weeks, months, or years.

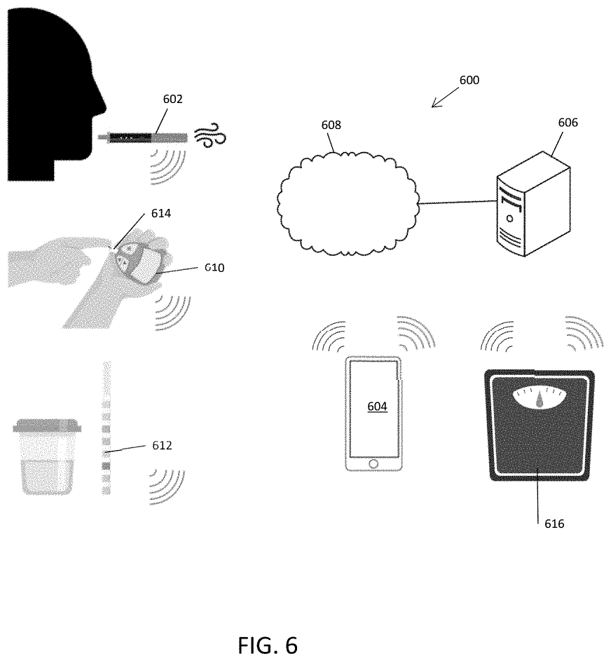

FIG. 6 illustrates a system 600 for monitoring and managing a personalized lifestyle program of a user, according to some embodiments. System 600 includes a ketone analyzer which may be a breath analyzer 602 for measuring the amount of ketone in a user's breath, a blood analyzer 610 for measuring the amount ketone in a user's blood, or a urine strip 612 for measuring the amount of ketone in a user's urine, and a portable electronic device 604 for monitoring and managing the user's ketosis levels using the ketone measurements from the breath analyzer 602, blood analyzer 610, or urine strip 612. It is not required for all ketone measurement types to be utilized. For example, some embodiments may only include a breath analyzer, but not blood or urine analysis.

The breath analyzer 602, blood analyzer 610, and urine analyzer (e.g. urine strip 612) can be a portable device or strip that the user can take with them and use repeatedly throughout the day for measuring acetone levels at different times The portable electronic device 604 can be a smartphone, tablet, laptop, smartwatch, or any other suitable device that may run a ketosis monitoring software application (App) for receiving acetone measurements from the breath analyzer 602 and using the measurements for assessing the user's level of ketosis.

A scale for weight measurement 616 may be used as a program task, which may communicate wirelessly with the App

The App can provide useful information to the user to help the user track and manage their eating choices and exercise goals and to perform previously described program tasks. Such information can include current ketosis level, which can help a user gauge whether a desired ketosis level is being achieved. According to some embodiments, the App can provide dietary and lifestyle recommendations for achieving ketosis level goals. Recommendations can be generated based on the acetone measurements and based on user-specific factors such as user attributes (e.g., weight, dietary preferences, duration on program) and user goals (e.g., weight loss, weight maintenance).

The App can provide users with the ability to receive points or virtual currency for performing program tasks.

In some embodiments, the portable electronic device 604 is communicatively connected to a server system 606 (e.g., a cloud service) via one or more networks 608. User information may be stored in one or more databases associated with the server system 606. The App can communicate the user's level of ketosis to the server system 606. The server system 606 can generate recommendations, points or virtual currency, for the user and transmit the recommendations, points or virtual currency earned, to the App for display to the user. The server system 606 can facilitate other functionality of the App, including tracking of previously earned points or virtual currency, social media related capabilities for interconnecting a community of App users.

To measure the amount of ketone in the user's breath, the user blows into the breath analyzer 602. The ketone detected in breath may be acetone.

To measure the amount of ketone in a user's blood, the user obtains a sample of blood on a blood test strip 614 for interpretation by the blood analyzer 610. The analyzer may also have a mechanism that continuously monitors the level of ketones in a user's interstitial fluid using a sensor that is implanted under the skin (not shown). The ketone detected may be β-hydroxybutyrate.

To measure the amount of ketone in a user's urine, the user collects a sample of urine and places the urine on a urine strip 612 for interpretation. The interpretation may be done visually by the user, or may be performed by an analyzer (not shown).

As discussed in more detail below, a sensor in the breath analyzer 602 generates a signal that is based on the amount of acetone in the user's breath. The breath analyzer 602 converts the signal to one or more digital measurements that are provided to the portable electronic device 604. The breath analyzer 602 and portable electronic device 604 can communicate via a wired or wireless connection. The user may also manually input a breath acetone level. In an example, the breath analyzer 602 and portable electronic device 604 communicate wirelessly (e.g., via Bluetooth™, ZigBee™, Institute of Electronic and Electric Engineers (IEEE) (TM) 802.11x, etc.). Once connected to the portable electronic device 604, the breath analyzer 602 can send acetone measurements to the portable electronic device 604.

Blood is placed on a blood test strip 614 which is then inserted into a blood analyzer 610 which is capable of determining the concentration of ketones, including β-hydroxybutyrate or other ketone such as acetoacetate, through a range of technologies. One example of such sensing system would be electrochemical detection of β-hydroxybutyrate in blood. One example of such a system is commercially available from Abbott Laboratories, Inc. of Chicago, Ill. This may also be done using a continuous monitor which includes a sensor that is implanted under the skin and samples interstitial fluid (not shown). The sensor can measure the concentration of ketones in blood or interstitial fluid, and can communicate with portable electronic device 604 via a wired or wireless connection. The user may also manually input a blood ketone level. In an example, the blood analyzer 610 and portable electronic device 604 communicate wirelessly (e.g., via Bluetooth, ZigBee, IEEE 802.11x, etc.). Once connected to the portable electronic device 604, the blood analyzer 610 can send ketone measurements to the portable electronic device 604.

Chemicals in the urine strip 612 react with acetoacetate in the urine which indicates the level of acetoacetate or other ketone such as β-hydroxybutyrate. This may be through a change in the color of the reactive portion of the urine strip 612. The urine strip 612 may connect to a urine analyzer (not shown) which may communicate to a portable electronic device 604 via a wired or wireless connection. The user may also manually input a urine ketone level. In an example, the urine analyzer and portable electronic device 604 communicate wirelessly (e.g., via Bluetooth, ZigBee, IEEE 802.11x, etc.). Once connected to the portable electronic device 604, the urine analyzer can send ketone measurements to the portable electronic device 604.

In some embodiments, the App can provide guidance to the user on using the breath analyzer 602, blood analyzer 610, or urine analyzer. This guidance can be based on data received from the breath analyzer 602, blood analyzer 610, or urine analyzer. For example, the breath analyzer 602 may send an indication to the portable electronic device 604 that the breath analyzer is ready for use, and the App may display a notification to the user that the user can begin blowing into the breath analyzer.

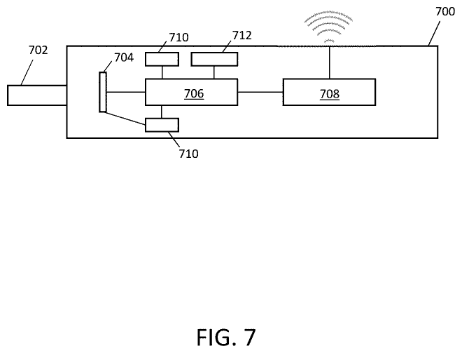

FIG. 7 illustrates an exemplary breath analyzer 700, according to one embodiment. Breath analyzer 700 includes a breath inlet 702 for receiving a user's breath, a sensor 704 for sensing an amount of acetone in the breath received through the breath inlet 702, one or more processors 706 for processing signals from the acetone sensor 704 to generate acetone measurements, and a communication interface 708 for communicating (e.g., wirelessly) with a remote device, such as portable electronic device 604 of FIG. 6, to provide the acetone measurements.

In some embodiments, the breath analyzer 700 is a handheld device. The user can bring the device up to the user's mouth to blow into the breath inlet 702. In other embodiments, the breath analyzer is a table-top device or includes a table-top portion in addition to a separate handheld portion for blowing into. The breath analyzer 700 may be a portable device that a user can take with them, for example, to use outside of the home. A handheld, portable breath analyzer enables a user to have the analyzer with them throughout the day for taking multiple times in measurements in a day and can ensure regular use.

In use, a user exhales into the breath inlet 702. The breath flows over the acetone sensor 704. The acetone sensor 704 generates a signal that is proportional to the amount of acetone in the breath flowing over the acetone sensor 704. The processor 706 generates one or more measurements from the signal generated by the sensor. The communication interface 708 establishes a communication connection with a portable electronic device, such a user's smartphone, or to a personal computer or other computing device, and transmits one or more of the measurements to the external device.

The acetone sensor 704 is configured to sense the amount of acetone in the breath of the user. Any suitable sensor or sensing system may be used. In some embodiments, the acetone sensor 704 is a metal oxide semiconductor sensor that is specific for acetone. The sensor may be made of tungsten, tin, aluminum, silicon, silicone, carbon, oxygen, and other metals and compounds, and configured so that the surfaces of the metal oxide nanoparticles react with oxygen from the air and reducing gases such as acetone, resulting in conductivity changes that are measured. The level of conductivity change produced during a breath measurement is turned into a digital signal. This digital signal is received by a processor 706, and transmitted by a communication interface 708 to the app.

In some embodiments, the sensor may be an optical or chemical based sensor. In some embodiments, the breath sample may be collected in a removable container or bag and analyzed by an analyzer separate from the removable container or bag (not shown).

Returning to FIG. 7, in some embodiments, Processor 706 converts the analog current signals from the sensor into digital measurements of the amount of acetone in the user's breath, using well-known analog to digital conversion techniques and hardware. The result of the digital conversion is often referred to herein as raw data. In some embodiments, the one or more processors transform the raw data into the one or more measurements, such as for filtering noise or removing a sensor offset. In some embodiments, the measurements are uncalibrated such that they include sensor-specific biases. The uncalibrated measurements are transmitted to the external device and calibration of the measurements is performed by the external device.

In some embodiments, breath analyzer specific calibration parameters are stored on a memory 712 of the analyzer and communicated to the external device for calibration of the measurements by the external device. Analyzer specific calibration parameters are parameters that have been determined for the specific analyzer, such as through laboratory or factory testing of the analyzer before delivery to a user. Analyzer specific calibration parameters can be generated, for example, by exposing the breath analyzer to a known concentration of acetone and determining the difference between the data generated by the analyzer and the known concentration. Analyzer specific calibration parameters are those that will adjust the analyzer data to the known concentrations, according to one or more predetermined conversion functions. For example, in some embodiments, measurements generated from the sensor signal may have an offset and the calibration parameter(s) provided to the external device may include the amount of the offset. The external device may subtract the offset from the acetone measurements received from the analyzer to generate calibrated measurements.

In some embodiments, the processor 706 may calibrate the measurements so that calibrated measurements are provided to the external device. Using the example above, the processor 706 may use the amount of the offset to adjust the measurements generated by the raw conversion of the sensor signals.

Calibration parameter(s) may be determined at the factory by exposing the acetone sensor 704 to one or more levels of known concentrations of acetone and determining a difference between the raw data from the sensor and the known concentration. One or more calibration parameters may be generated based on the difference and these parameters may be stored in the memory 712 for use by the processor 706 for generating calibrated measurements or for transmission to the external device along with the raw data for calibration of the data by the external device. The calibration parameter(s) may also be stored in the server system, and used by the external device along with raw data to produce a calibrated measurement.

The communication interface 708 provides a communication connection with an external device for communicating the acetone measurements and any other relevant information to the external device. The communication interface 708 may be a wireless network interface that may be coupled to a wireless antenna for communicating acetone measurements wirelessly to the portable electronic device (or any other external device).

In some embodiments, the breath analyzer includes a display 710. The display 710 can be used to communicate information to the user. In some embodiments, the display 710 is used to display analyzer status information. The display 710 can be one or more indicator lights, such as light emitting diodes (LEDs), that indicate analyzer status. The display 710 can be or include a liquid crystal display (LCD) screen for displaying numbers or text. In some embodiments, one or more lights are used to indicate analyzer status, such as a ready light for indicating that the analyzer is not ready for use, and/or a green light to indicate that the analyzer is ready. A series of multiple lights may be used to indicate progress to a state, such as progress to a ready-to-use state or progress to a measurement cycle completion state. In some embodiments, one or more indicators are used to indicate battery life, an on/off state, and/or a wireless connection state. In some embodiments, an LCD screen may be used to provide any relevant information, including states of the device (on/off, ready-for-use, low battery, etc.), and acetone measurements.

The breath analyzer 700 may include one or more additional sensors 714 for facilitating acetone measurements. In some embodiments, the breath analyzer includes a temperature sensor that can be used for managing a temperature sensitivity of the acetone sensor. In embodiments in which the signal generated by the acetone sensor varies with temperature of the sensor, the temperature sensor may be used to monitor the temperature of the breath analyzer. Temperature data can be used to indicate to the user when the analyzer is ready for use and/or can be used to adjust the acetone measurements. The monitored temperature may be used to notify a user when the analyzer is ready for use. For example, the breath analyzer may send an indication to the portable electronic device that the breath analyzer is ready for use based at least in part of the sensor reaching a predetermined temperature. In some embodiments, the breath analyzer 700 includes a heating system for controlling the temperature of the acetone sensor 704.

In some embodiments, the analyzer does not have a temperature sensor, but may still account for a temperature sensitivity by waiting for a predetermined time after start-up before indicating to the user (e.g., via the display 710 or via an indication sent to the portable electronic device) that the analyzer is ready to use. For example, it may be determined that the analyzer will warm to a satisfactory temperature in a predetermined period of time, and the display 710 may indicate that the analyzer is ready to use only after the predetermined time has elapsed or the breath analyzer may transmit a ready to use signal to the portable electronic device only after the predetermined time has elapsed.

In some embodiments, the breath analyzer includes a flow sensor for sensing whether a user is blowing into the breath inlet 702. The flow sensor may be any suitable type of sensor for sensing that a user is blowing into the analyzer. In some embodiments, the flow sensor is a pressure sensor that senses the pressure of the breath created by the user exhaling. In other embodiments, the flow sensor includes a turbine that rotates in response to fluid flow. In some embodiments, data from the flow sensor (e.g., pressure sensor) may be transmitted to the portable electronic device as an indication of the usage of the breath analyzer. In other embodiments, the breath analyzer may use the data to generate and usage status indication, which is then provided to the portable electronic device and/or displayed via display 710.

As stated above, the breath analyzer, such as breath analyzer 700, communicates with a portable electronic device, such as a smartphone, tablet, or smartwatch, executing an App. According to some embodiments, the App can assist the user in using the breath analyzer, can determine a ketosis score that reflects the level of ketosis of the user based on measurements from the breath analyzer, and can provide rewards to the user based on the ketosis score.

Returning to FIG. 1, at step 116 the result of program tasks performed may be displayed to the user on the App. This may include a calculated score, numerical values, grades, or qualitative descriptions.

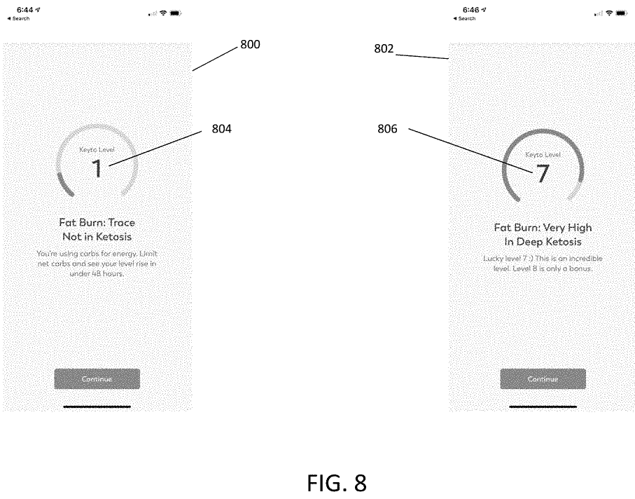

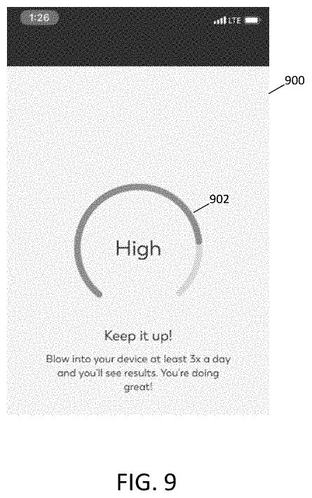

In some embodiments, at step 116, the App may display the score associated with the ketone measurement to the user. For example, the App may display a qualitative score of “moderate” or a quantitative score of “3” to the user. This can help a user understand their body's current ketosis level. A user can learn from the ketosis score how the user's diet affects the level of ketosis and how the level of ketosis changes throughout the day and over days, weeks, and months. An exemplary ketosis score display user interface 800, and user interface 802 is illustrated in FIG. 8 with a ketosis score 804 of “1” displayed and with a ketosis score 806 of “7” displayed. An exemplary ketosis score display user interface 900 is illustrated in FIG. 9 with a ketosis score 902 of “High” displayed.

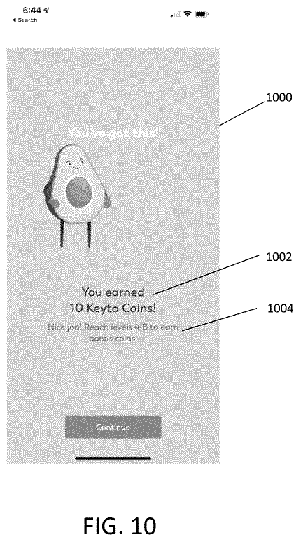

The App may use the ketone measurement or ketosis score or other program tasks to award the user points or virtual currency, which are then displayed to the user. An exemplary virtual currency award display user interface 1000, is illustrated in FIG. 10 with “10 Keyto Coins” 1002 displayed. In 1002, “Keyto Coins” are an example virtual currency.

Points and virtual currency may include numerical values, letters, percentages, real currencies, cryptocurrencies, imaginative currencies, grades, descriptions, stock shares, and other scales that span from lesser to greater.

According to some embodiments, additional points or virtual currency may be awarded based on the ketosis score with higher scores receiving more points or virtual currency. This is illustrated in FIG. 10. with “Reach levels 4-8 to earn bonus coins” 1004. In this example, “coins” are a virtual currency.

The number of points or virtual currency awarded may vary with from low values to very high values. The number of points awarded for a measurement or ketosis score may be pre-determined or include a random component that changes the points or virtual currency rewarded for the same ketosis score.

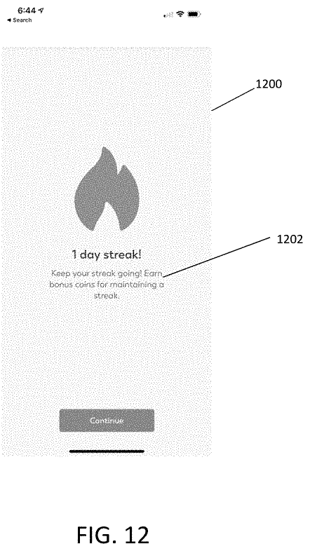

According to some embodiments, the user may gain additional points or virtual currency for maintaining a consecutive daily streak of performing ketone measurements. This is illustrated in FIG. 12. An exemplary user interface 1200 shows that users may “Earn bonus coins for maintaining a streak” 1202. In this example, “coins” are a virtual currency.

Going back to FIG. 1, in some embodiments, at step 114, the App may display program tasks to the user. The user may earn points or virtual currency through completion of tasks for compliance or assessment.

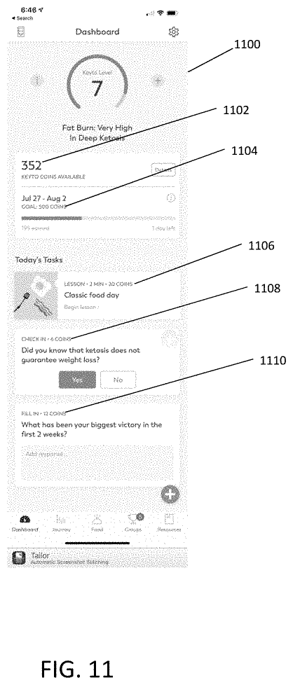

An exemplary display user interface 1100, is illustrated in FIG. 11 with examples of program tasks that a user can perform on the App. Completing a lesson 1106, completing a check question in 1108, and completing a fill in question 1110 are displayed. In this example the user would receive 20 “Keyto Coins,” an exemplary virtual currency for completing the lesson 1106, 6 “Keyto Coins” for completing the check in question, and 12 “Keyto Coins” for completing the fill in question.

In some these embodiments, the user may redeem points or virtual currency for virtual rewards such as badges, rankings, and software features. The user may also redeem points or virtual currency or tangible rewards such as money, gift cards, or other prizes. An exemplary display user interface 1100, is illustrated in FIG. 11 with the number of “Keyto Coins,” an example virtual currency, available to the user for redeeming for virtual or tangible rewards is shown in 1102.

In some embodiments, the App may give the user a personalized or standard goal for points or virtual currency to be earned in a given time period which may include days, weeks, months, or years. An exemplary user display goal 1104 is shown in FIG. 11.

In some embodiments, after the tolerability score is determined at step 118 from FIG. 1, it may be determined if the instructions are tolerable step 120 or not tolerable by the user step 122. The user may also optionally repeat a heart failure or metabolic disease assessment step 124.

In some embodiments, the program recommendations may be updated at step 126, based on the tolerability scores, whether the instructions were tolerable by the user, or the results from the repeat heart failure or metabolic assessments. The results of the update may be displayed to the user step 108. This process may be repeated as previously described and as shown in the flow diagram in FIG. 1.

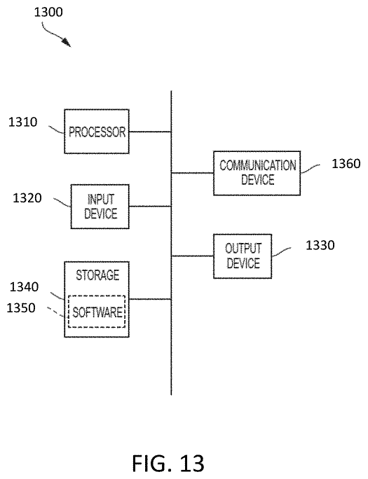

Computing device 1300 can be a host computer connected to a network. Computing device 1300 can be a client computer or a server. As shown in FIG. 13, computing device 1300 can be any suitable type of microprocessor-based device, such as a personal computer, workstation, server, or handheld computing device, such as a smartphone, a tablet, or a smartwatch. The computer can include, for example, one or more of processor 1310, input device 1320, output device 1330, storage 1340, and communication device 1360. Input device 1020 and output device 1030 can generally correspond to those described above and can either be connectable or integrated with the computer.

Input device 1320 can be any suitable device that provides input, such as a touch screen or monitor, keyboard, mouse, or voice-recognition device. Output device 1330 can be any suitable device that provides output, such as a touch screen, monitor, printer, disk drive, or speaker.

Storage 1340 can be any suitable device that provides storage, such as an electrical, magnetic, or optical memory, including a RAM, cache, hard drive, CD-ROM drive, tape drive, or removable storage disk. Communication device 1360 can include any suitable device capable of transmitting and receiving signals over a network, such as a network interface chip or card. The components of the computer can be connected in any suitable manner, such as via a physical bus or wirelessly. Storage 1340 can be a non-transitory computer readable storage medium comprising one or more programs, which, when executed by one or more processors, such as processor 1310, cause the one or more processors to perform methods described herein, such as method 100 of FIG. 1.

Software 1350, which can be stored in storage 1340 and executed by processor 1310, can include, for example, the programming that embodies the functionality of the present disclosure (e.g., as embodied in the systems, computers, servers, and/or devices as described above). In some embodiments, software 1350 can include a combination of servers such as application servers and database servers.

Software 1350 can also be stored and/or transported within any computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1340, that can contain or store programming for use by or in connection with an instruction execution system, apparatus, or device.

Software 1350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Computing device 1300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, Digital Subscriber Line (DSL), or telephone lines.

Computing device 1300 can implement any operating system suitable for operating on the network. Software 1350 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The methods and systems described above can help a user prevent or treat heart failure with persevered ejection fraction by giving them personalized instructions based on tolerability to previous instructions. The user can be provided with metrics of the user's level of ketosis, weight, activity, quiz scores, and other scores, helping a user stay motivated and informed.

In addition to heart failure with preserved ejection fraction, in some examples the disease being treated with this system or method may be other forms of heart failure, atrial fibrillation, coronary artery disease, stroke, dyslipidemia, hypertension, non-alcoholic fatty liver disease (NAFLD) non-alcoholic steatohepatitis (NASH), polycystic ovarian syndrome (PCOS), Type 1 diabetes, Type II diabetes, kidney disease, pre-diabetes, metabolic syndrome, obesity, epilepsy, Alzheimer's disease, or other neurologic or psychiatric diseases.

An example of how a system described in the foregoing description can work in practice was outlined in a publication: Keyto app and device versus WW app on weight loss and metabolic risk in adults with overweight or obesity: A randomized trial (1), which is herein incorporated by reference in its entirety. In that study, participants in the Keyto program were given initial assessments which included blood tests (hemoglobin A1C, ALT, LDL, HDL, Triglycerides, CRP, Homocysteine, 1p(a), etc.) as well as a weight measurement through a wireless bluetooth scale. They were then given a personalized program, delivered through an app, with specific tasks to be completed. The app also included educational lessons, podcasts, and other multimedia. For feedback, users were also given a breath acetone sensor, which provided them information on a 1-8 scale for if they were in ketosis. This system proved to be significantly more effective than the comparator group, the WW app, for both weight loss and metabolic markers such as Hemoglobin A1C and ALT.

The foregoing description, for the purpose of explanation, has been described with reference to specific embodiments. However, the illustrative discussions above are not intended to be exhaustive or to limit example embodiments to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

The various embodiments presented above are merely examples and are in no way meant to limit the scope of this disclosure. Variations of the innovations described herein will be apparent to persons of ordinary skill in the art, such variations being within the intended scope of the present disclosure. In particular, features from one or more of the above-described embodiments may be selected to create alternative embodiments comprises of a sub-combination of features which may not be explicitly described above. In addition, features from one or more of the above-described embodiments may be selected and combined to create alternative embodiments comprised of a combination of features which may not be explicitly described above. Features suitable for such combinations and sub-combinations would be readily apparent to persons skilled in the art upon review of the present disclosure as a whole. The subject matter described herein intends to cover and embrace all suitable changes in technology.

The following Citations are herein incorporated by reference in their entirety:
1 Falkenhain K, Locke S R, Lowe D A, Reitsma N J, Lee T, Singer J, Weiss E J, Little J P. Keyto app and device versus WW app on weight loss and metabolic risk in adults with overweight or obesity: A randomized trial. Obesity (Silver Spring). 2021 October; 29(10):1606-1614. doi: 10.1002/oby.23242. Epub 2021 Sep. 14. PMID: 34124856; PMCID: PMC8518592.
2 Locke S R, Falkenhain K, Lowe D A, et al. Comparing the Keyto App and Device with Weight Watchers' WW App for Weight Loss: Protocol for a Randomized Trial. JMIR Res Protoc. 2020; 9(8):e19053. Published 2020 Aug. 17. doi:10.2196/19053.

1. A system for promoting improvement of heart failure with preserved ejection fraction, the system comprising:
one or more processors configured to:
generate, based on a heart failure assessment, a lifestyle program for the promoting improvement of heart failure with preserved ejection fraction including one or more program instructions;
generate, based on the one or more program instructions, one or more user interface screens for performing the one or more program instructions through one or more program tasks; and
determine a user tolerance of the lifestyle program based on compliance to the performing the one or more program instructions through the one or more program tasks.

2. The system of claim 1, wherein the heart failure assessment is performed using the one or more processors.

3. The system of claim 1, wherein the performing the heart failure assessment includes receiving heart failure assessment results.

4. The system of claim 1, wherein the one or more processors is configured to update the lifestyle program to be more or less strict based on the user tolerance.

5. The system of claim 1, wherein the heart failure assessment includes a ketone measurement.

6. The system of claim 1, wherein the heart failure assessment includes measurement of a severity of the heart failure with preserved ejection fraction as measured by a patient reported outcome survey.

7. The system of claim 6, wherein the heart failure assessment includes a health status test.

8. The system of claim 7, wherein the health status test includes Kansas City Cardiomyopathy Questionnaire, Minnesota Living with Heart Failure (MLHF) survey, or other surveys that assess symptoms, quality of life, exercise tolerance, demographics, or personal preferences.

9. The system of claim 1, wherein the heart failure assessment includes a medical diagnostic test.

10. The system of claim 9, wherein the medical diagnostic test includes an echocardiogram, exercise tolerance test, magnetic resonance image (MRI), or a blood test that includes blood glucose level, Homeostatic Model Assessment of Insulin Resistance (HOMA-IR), insulin level, B-type natriuretic peptide (BNP) level, N-terminal BNP (nt-BNP) level, lipid level, liver function test level, or ketone level.

11. The system of claim 1, wherein the program instructions include: food intake instructions.

12. The system of claim 11, wherein the food intake instructions comprise ketogenic nutrition instructions, low-carb nutrition instructions, Mediterranean diet based instructions, reduction in refined carbohydrates and sugar instructions, eating window instructions comprising prolonged fasting, intermittent fasting, or no restriction to feeding window instructions.

13. The system of claim 1, wherein the program instructions include exercise or activity instructions comprising: i) intensive exercise or intensive activity, ii) moderate exercise or moderate activity, iii) mild exercise or mild activity, or iv) no exercise or no activity.

14. The system of claim 1, wherein an expected effect of the lifestyle program is estimated, and displayed on a display.

15. The system of claim 1, wherein the determining of the user tolerance includes a second heart failure assessment, which is a same type of assessment as the heart failure assessment.

16. The system of claim 1, wherein the determining of the user tolerance includes a second heart failure assessment, which is different than the heart failure assessment.

17. The system of claim 1, wherein one of the program tasks includes a ketone measurement.

18. The system of claim 17, wherein the ketone measurement is performed by an analyzer, wherein the analyzer is a breath ketone analyzer, a blood ketone analyzer, or a urine ketone analyzer.

19. The system of claim 18, wherein the one or more processors communicate wirelessly with the analyzer.

20. The system of claim 17, wherein the one of the program tasks includes determining a ketosis score based on the ketone measurement, and one or more thresholds that are associated with levels of ketosis in fluid, wherein the ketosis score is an estimate of a level of ketosis.

21. The system of claim 20, wherein the fluid is breath, blood, or urine.

22. The system of claim 1, wherein the determining of the user tolerance includes compliance to and results from at least one of the program tasks, which include ketone measurement, weight loss, activity, user response to questions, completion of lessons, one or more quizzes, user response to assessment tasks, or the heart failure assessment.

23. The system of claim 1, wherein the lifestyle program includes one or more rewards for completion of one or more of the program tasks.

24. The system of claim 23, wherein the one or more rewards are for one or more of the program tasks which include: performing ketone measurements, answering questions, completing assessments, or losing weight.

25. The system of claim 23, wherein the one or more rewards are awarded for performing a ketone measurement with a ketone analyzer, wherein the reward includes points, virtual currency, numerical values, letters, percentages, real currencies, cryptocurrencies, imaginative currencies, grades, qualitative descriptions, stock shares, prizes, or scales that span from lesser to greater.

26. The system of claim 24, wherein the processor is configured to permit a user to redeem points or virtual currency earned from compliance to and results from the one or more program tasks for prizes.

27. The system of claim 26, wherein the prizes include badges, rankings, software features, or tangible prizes.

28. The system of claim 26, wherein the tangible prizes include money or gift cards.

29. The system of claim 1, wherein tolerability scores are calculated based on the compliance to and results from the program tasks.

30. The system of claim 1, wherein face-to-face, telephone, or virtual consultation with a user is used as inputs to tolerability scores.

31. The system of claim 1, wherein the generation of program instructions is based on algebraic functions, stochastic functions, decision tree protocols, or machine learning techniques which include neural networks, random forest, collaborative filtering, or support vector networks.

32. The system of claim 1, wherein the program instructions includes the use of exogenous ketones such as ketone salts or ketone esters, medium chain triglycerides, or short chain triglycerides.

33. The system of claim 1, wherein the lifestyle program is used for the treatment of the heart failure with preserved ejection fraction.

34. The system of claim 1, wherein a disease being treated further includes other forms of heart failure, atrial fibrillation, coronary artery disease, stroke, dyslipidemia, hypertension, non-alcoholic fatty liver disease (NAFLD), non-alcoholic steatohepatitis (NASH), polycystic ovarian syndrome (PCOS), Type 1 diabetes, Type II diabetes, kidney disease, pre-diabetes, metabolic syndrome, obesity, epilepsy, Alzheimer's disease, or other neurologic or psychiatric diseases.

35. A processor-implemented method for promoting improvement of heart failure with preserved ejection fraction, comprising:
generating, based on a heart failure assessment, a lifestyle program for promoting improvement of heart failure for with preserved ejection fraction including one or more program instructions;
generating, based on the one or more program instructions, one or more user interface screens for performing the one or more program instructions through one or more program tasks; and
determining a user tolerance of the lifestyle program based on compliance to the performing the one or more program instructions through the one or more program tasks.

36.-68. (canceled)

69. A non-transitory computer-readable medium which includes instructions that, when executed by one- or more processors, promotes improvement of heart failure with preserved ejection fraction, the instructions comprising:
instructions for generating, based on a heart failure assessment, a lifestyle program for promoting improvement of heart failure for with preserved ejection fraction including one or more program instructions;
instructions for generating, based on the one or more program instructions, one or more user interface screens for performing the one or more program instructions through one or more program tasks; and
instructions for determining a user tolerance of the lifestyle program based on compliance to the performing the one or more program instructions through the one or more program tasks.

70.-102. (canceled)
